(19) United States
(12) Patent Application Publication
(10) Pub. No.: US 2009/0076419 A1
(43) Pub. Date: Mar. 19, 2009

(54) LOSS-OF-BALANCE AND FALL DETECTION SYSTEM

(75) Inventors: Pavan K. Namineni, Ann Arbor, MI (US); Charles J. Jacobus, Ann Arbor, MI (US)

Correspondence Address: GIFFORD, KRASS, SPRINKLE,ANDERSON & CITKOWSKI, P.C, PO BOX 7021, TROY, MI, 48007-7021, US

(73) Assignee: Cybernet Systems Corporation, Ann Arbor, MI (US)

(21) Appl. No.: 12/126,502

(22) Filed: May 23, 2008

Related U.S. Application Data
(60) Provisional application No. 60/939,687, filed on May 23, 2007.

(51) Int. Cl. A61B 5/103

(52) U.S. Cl. 600/595

(57) ABSTRACT

A wireless monitoring/assessment system identifies and records selected basic activities of daily living, loss of balance, and falls of at-risk elderly using a minimum set of sensors. A wearable system collects data over an extended period from sensors mounted under each shoe/slipper inner sole, and three kinetic measurement sensors, one mounted on the subject's chest, and the other two on each thigh. The resulting data can be processed off line to determine if a loss of balance or fall has occurred and identify the basic activity that the subject was performing when it happened. The two inner sole foot pressure sensors provide data on the loading of the plantar aspect of each fore- and rear foot. Each kinematics measurement sensor module provides 3-axis acceleration and angular rate data from the torso and each thigh. All data is sampled at a high rate using an analog to digital converter, time stamped, and then stored in memory.

### REFERENCE TO RELATED APPLICATION

This application claims priority from U.S. Provisional Patent Application Ser. No. 60/939,687, filed May 23, 2007, the entire content of which is incorporated herein by reference.

### FIELD OF THE INVENTION

This invention relates generally to monitoring the movements of a human being and, more particularly, to methods and systems for studying and determining a patient's activity, a loss of balance, or a fall event.

### BACKGROUND OF THE INVENTION

There are many devices that are designed to determine a subject's physical position or movement. The field may be segmented into (a) devices that detect an event only and provide some kind of action in response to the event, and (b) devices that log data for the purposes of study and prediction as well as provide real-time event detection. U.S. Pat. No. 7,248,172 is an example of the former and U.S. Pat. No. 6,834,436 is an example of the latter.

Physically, such devices fall into two categories. The first category is a sensor module that is worn somewhere on the body and transmits to a remote location when an event has been detected (as described in U.S. Pat. No. 7,248,172). The second would be a hardwired personal area network (as described in U.S. Pat. No. 6,834,436).

The basic purpose of these devices is to (a) monitor kinematic output signals from a subject (as described in U.S. Pat. No. 7,095,331) (b) use the signal data to study the movements of a subject or determine a fall event (as described in U.S. Pat. No. 7,141,026), and (c) notify emergency response personnel of a potentially harmful event (as described in U.S. Pat. No. 6,433,690).

These systems use various sensor modules to monitor and study kinematic output signals. Most devices include an accelerometer to make these measurements (as described in U.S. Pat. No. 6,997,882). Others combine accelerometers with gyroscopes (as described in U.S. Pat. No. 7,141,026). A third type of device uses insole foot sensors only to determine patient movement (as described in U.S. Pat. No. 6,183,425).

As is apparent from above, there is no device that (a) provides the detailed data to allow the study of system inputs so that a fail can be predicted and potential preventative measures can be taken, (b) uses a wireless personal area network to eliminate all wires from the system, and (c) combines accelerometer, gyroscope, and foot force data.

Combining these features into multiple small low profile modules that are comfortable to wear will be state-of-the-art in monitoring and studying the stability of elderly patients. Therefore prior units were not built with highly detailed data collection combined with the comfort that a truly wireless system provides.

### SUMMARY OF THE INVENTION

This invention is directed to a loss-of-balance detection system and method. It is designed to create a monitoring/assessment tool to determine if assisted living is necessary for an elderly person based on loss of balance when performing everyday activities. In the preferred embodiment the system is wireless and includes a base station, 3-kinematics measurement sensors, and 2-foot force sensors. The base station is connected to a computer where it initializes the wireless network and uploads the system data at the conclusion of the test. The 3-kinematics measurement sensors are attached to the chest and both thighs. The 2-foot force sensors are worn in the insole of the shoes. All these modules are designed to sample data at high rates and store the data on a module memory card.

The invention can also be used as wireless fall event detection system. If the system is going to be used to prevent and detect falls in real time the wireless feature may be utilized with a lower sampling rate to allow for processing. The sensor modules wirelessly transmit the data to a base station where processing of the data is preformed. If a fall event is detected then the base station will alert emergency personnel.

The system is designed to be non-invasive to increase the validity of the data. A wireless personnel area network is used so that there are no wires in the wearable portion of the system. This is to remove the restrictive nature of wires hanging on the body and in return create more mobility for the subject under examination.

The combination of sensors is unique because they combine acceleration, angular velocity, and foot force data all in one system. Previous systems have used at least one form of this data, but never has all three forms been combined in one system. To provide this data each kinematic measurement sensor module includes a 3-axis accelerometer for acceleration and a 3-axis gyroscope for angular velocity. The foot force sensors use two piezoresistive transducers each to produce foot floor pressure data.

### BRIEF DESCRIPTION OF THE DRAWINGS

FIG. 1 shows an overall view of the system;

FIG. 2 is a block diagram of the Foot Force Sensor;

FIG. 3 is a block diagram of a Kinematics Measurement Sensor; and

FIG. 4 is a block diagram of the Base Station.

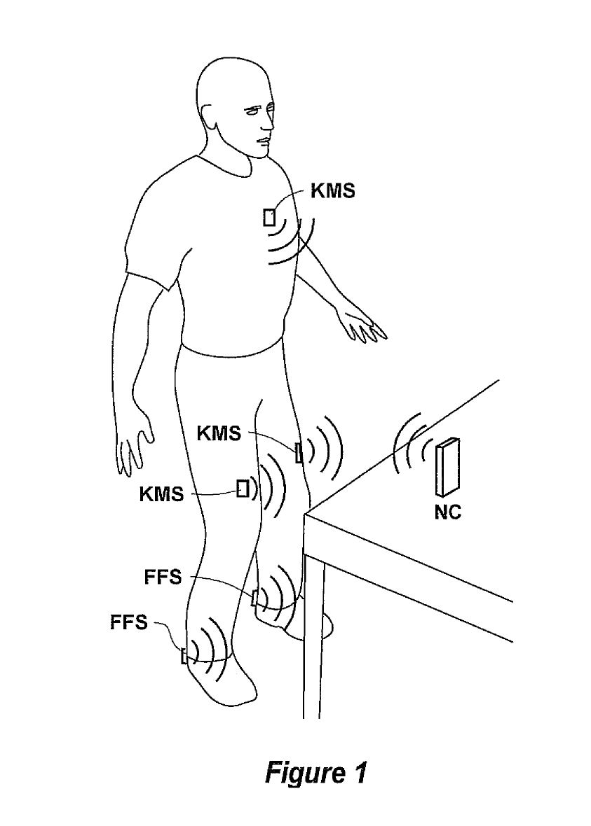

### DETAILED DESCRIPTION OF THE INVENTION

There are many applications that need methods and equipment to study and evaluate human movement. A few examples would include the running position of a track and field athlete, the proper lifting pattern of a mover, or to determine if someone's stability is potentially detrimental to their health. This invention is focused around the third application, but can be used for any of the mentioned or similar scenarios.

To support these applications, the disclosed system has the following features;

1. 2 foot force sensors

2. 3 kinematic measurement sensors

3. A base station

4. Portable system that can be worn anywhere

5. The ability to collect data at a high rate for extended periods of time

6. Raw data access

7. Removes the constricted feeling of a wire system

8. Small sensor packaging to increase the comfort of the system

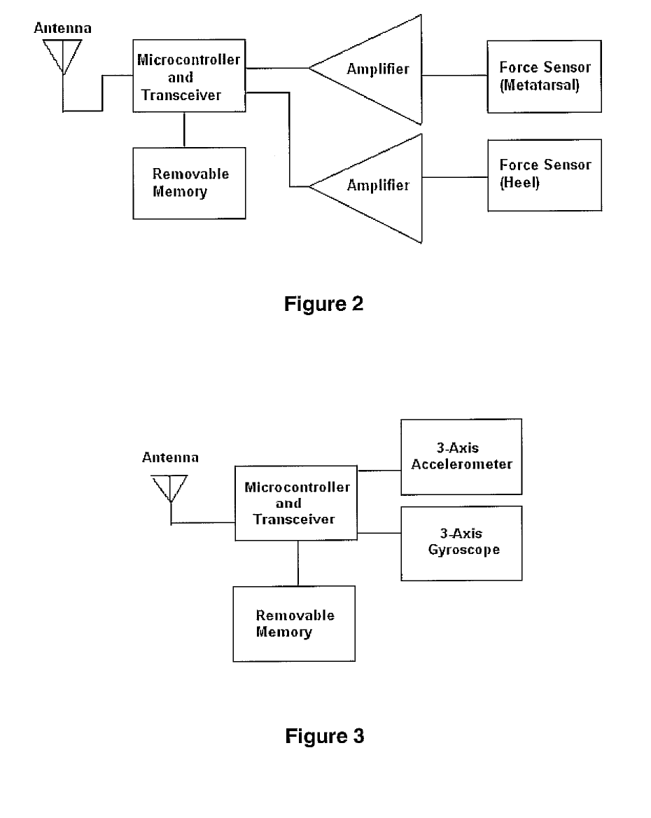

The foot force sensor (FFS) measures the ground reaction between the foot and the floor. The force-sensing transducers are preferably located in the insole under the heel and the second metatarsal head of each foot in order to monitor ground reaction force magnitude. They are connected to amplifiers that condition the signal and then send it to a microprocessor where it is stored in memory or wirelessly transmitted to the base station depending on the application. All the components except the transducers are housed in a module that is strapped around the ankle. The module features a transceiver and printed circuit board (PCB) antenna for synchronization with the rest of the system or data upload with the base station. A block diagram of the system is shown in FIG. 2.

The kinematics measurement system (KMS) measures the angular rate, acceleration, and angle of the area of interest about 3 orthogonal axes. In the preferred embodiment each KMS uses a 3-axis accelerometer and 3-axis gyroscope to determine these measurements. These components are connected to a microprocessor, transceiver, external memory, and a printed circuit board (PCB) antenna to complete the circuitry. The KMS modules are preferably worn on the chest and both thighs though more or fewer may be used, each with one or both of the accelerometers and gyroscopes, depending upon the patient and the activity to be monitored. A block diagram of the system can be viewed in FIG. 3.

A kinematics measurement system (KMS) module is strapped to the chest of the subject using a harness. With this module on the chest, determine activities like falling, bending, twisting, and turning are possible.

To improve the input data for determining loss of balance, one kinematics measurement system (KMS) module is placed on each thigh of a subject using a harness. The modules assist in understanding the position and the activity of the person wearing the unit. The information is invaluable to understanding of what causes a loss of balance. The modules are used to differentiate sitting from standing and to provide information on pedal activities like waddling in place that can't be determined with a chest module only.

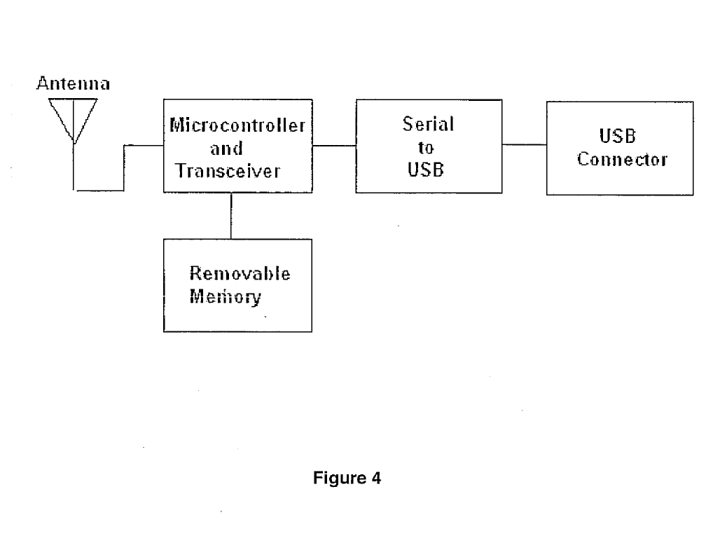

The purpose of the base station (BS) module is to initialize the network, provide an accurate time stamp, and read the data from the external memory cards or receive the data wirelessly depending on the application. The module connects to a computer to get an accurate time stamp and to transfer the data from the memory card or wireless network to the computer. The timestamp is broadcast to the modules during initialization of the network and attempts to update periodically if the system is within range until the test is complete. A block diagram of the system is depicted in FIG. 4.

An important aspect of the system architecture is the portability of the system. The system does not need to be in range of the base station to function. The system only needs to be started in the presence of the base station that also functions as a coordinator of the wireless network. After the base station has started the network, it will synchronize the sensors. Once the synchronization has occurred the base station is no longer necessary until the studies have finished.

The system has been designed to collect data at a high sampling rate for studying purposes. To accommodate a high sampling rate, the upload of data through a wireless network has to be eliminated. The data has to be stored directly on the sensor module. To provide this functionality each sensor module is equipped with a micro SD removable memory card. The memory card is sized to provide 24 hours of data collection.

The system provides access to the raw data that has been collected from the system. This allows the collector access to the true data and can filter, average, or sample the data per the desires of the study.

The system removes the constricted feeling of wires that previous systems have been unable to avoid. It uses a wireless personal area network to keep the individual modules in synchronization and each device has its own battery source to power the module.

Small non-invasive packaging is used for the sensor modules. This is possible because each sensor module uses a PCB antenna for communication, which eliminates an awkward external antenna. The kinematic measurement system utilizes state of the art packaging for the 3-axis accelerometer and 3-axis gyroscope that combines all three axes in one surface mount component.

We claim:

1. A loss-of-balance detection system, comprising:
two foot-force sensors, each supported under a respective foot of a wearer;
at least one sensor module containing a kinematics measurement sensor coupled to a portion of the body of a wearer; and
a processor operative to analyze information received from each of the sensors and determine whether the wearer is experiencing a loss of balance or has experienced a fall based upon one of both of the following:
a) one or both feet of the wearer have ceased making ground contact for a predetermined period of time in accordance with information received from the foot-force sensors; and
b) a portion of the wearer's body is experiencing a movement or is in a position contrary to normal activity in accordance with information received from the kinematics measurement sensor.

2. The loss-of-balance detection system of claim 1, wherein each foot force sensor is comprised of two piezoresistive transducers that are placed under the heel and metatarsal region of a foot to measure the ground reaction between the foot and the floor of the heel and metatarsal regions.

3. The loss-of-balance detection system of claim 1, wherein the kinematics measurement sensor is a 3-axis accelerometer.

4. The loss-of-balance detection system of claim 1, wherein the kinematics measurement sensor is a 3-axis gyroscope.

5. The loss-of-balance detection system of claim 1, wherein each foot-force sensor and the sensor module is in wireless communication with a base station in communication with the processor.

6. The loss-of-balance detection system of claim 1, wherein information from the foot force sensors and sensor module are time-stamped for synchronization purposes.

7. The loss-of-balance detection system of claim 1, wherein the foot force sensors and sensor module each include a memory for local storage of information provided to the processor.

8. The loss-of-balance detection system of claim 7, wherein each memory is disposed on a removable memory card that can be plugged into a separate unit for data retrieval.

9. The loss-of-balance detection system of claim 1, further including a real-time fall event detector providing an emergency alert.

10. The loss-of-balance detection system of claim 1, wherein the sensor module is mounted to the chest of a wearer to determine if the wearer is falling, bending, twisting, or turning.

11. The loss-of-balance detection system of claim 1, including two sensor modules, one mounted on each leg of a wearer to differentiate sitting from standing or to provide information representative of pedaling.

12. A loss-of-balance detection system, comprising:
two foot-force sensors, each supported under a respective foot of a wearer;
three sensor modules, each including a 3-axis accelerometer and a 3-axis gyroscope, with one sensor module being torso-mounted to determine if the wearer is falling, bending, twisting, or turning, and one sensor module being coupled to each leg of a wearer to differentiate sitting from standing or to provide information representative of pedaling;
a base station is wireless communication with each sensor module; and
a processor in communication with base station to analyze information received from each of the sensors and determine whether the wearer is experiencing a loss of balance or has experienced a fall based upon one of both of the following:
c) one or both feet of the wearer have ceased making ground contact for a predetermined period of time in accordance with information received from the foot-force sensors; and
d) the angle, angular rate and acceleration of the torso and each leg about three orthogonal axes to determine if the wearer's body is experiencing a movement or is in a position contrary to normal activity in accordance with information received from each accelerometer and gyroscope.

13. The loss-of-balance detection system of claim 12, wherein each foot force sensor is comprised of two piezoresistive transducers that are placed under the heel and metatarsal region of a foot to measure the ground reaction between the foot and the floor of the heel and metatarsal regions.

14. The loss-of-balance detection system of claim 12, wherein information from each sensor is time-stamped for synchronization purposes.

15. The loss-of-balance detection system of claim 12, wherein each foot-force sensor and each sensor module includes a memory for local storage of information provided to the processor.

16. The loss-of-balance detection system of claim 15, wherein each memory is disposed on a removable memory card that can be plugged into a separate unit for data retrieval.

18. The loss-of-balance detection system of claim 12, further including a real-time fall event detector providing an emergency alert.
